(19) United States
(12) Patent Application Publication
(10) Pub. No.: US 2020/0155216 A1
(43) Pub. Date: May 21, 2020

(54) PRESSURE INHIBITOR FOR INTRAVASCULAR CATHETER SYSTEM

(71) Applicant: Boston Scientific Scimed Inc., Maple Grove, MN (US)

(72) Inventor: Eric A. Schultheis, San Clemente, CA (US)

(21) Appl. No.: 16/752,780

(22) Filed: Jan. 27, 2020

Related U.S. Application Data
(63) Continuation of application No. PCT/US2018/040984, filed on Jul. 6, 2018.
(60) Provisional application No. 62/537,898, filed on Jul. 27, 2017.

(51) Int. Cl. A61B 18/02; A61M 25/10

(52) U.S. Cl. CPC A61B 18/02; A61B 2018/00577; A61M 25/10186; A61M 25/1011

(57) ABSTRACT

A pressure inhibitor for an intravascular catheter system includes a check valve and/or a pressure relief valve. The intravascular catheter system includes a handle assembly, an inner balloon, an outer balloon and a low pressure fluid line. The inner balloon and the outer balloon define an inter-balloon space therebetween. The low pressure fluid line extends between the handle assembly and the inter-balloon spaced The low pressure fluid line is in fluid communication with the inter-balloon space. The pressure inhibitor is positioned on the low pressure fluid line. The pressure inhibitor inhibits flow of a fluid to the inter-balloon space. The pressure inhibitor can be positioned within the handle assembly or outside of the handle assembly. A method of inhibiting flow of a fluid to the inter-balloon space includes positioning a pressure inhibitor on the low pressure fluid of an intravascular catheter system.

### CROSS-REFERENCE TO RELATED APPLICATION

This application is a continuation of International Application No. PCT/US2018/040984, with an international filing date of Jul. 6, 2018, which claims priority on U.S. Provisional Application Ser. No. 62/537,898, filed on Sep. 7, 2017, and entitled “PRESSURE INHIBITOR FOR A CRYOGENIC BALLOON CATHETER SYSTEM”. As far as permitted, the contents of U.S. Provisional Application Ser. No. 62/537,898 are incorporated in their entirety herein by reference.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the normal conduction pattern of the heart. The energy delivery component of the system is typically at or near the most distal (farthest from the operator) portion of the catheter, and often at the tip of the device. Various forms of energy are used to ablate diseased heart tissue. These can include radio frequency (RF), balloon cryotherapy which uses cryoballoons, ultrasound and laser energy, to name a few. The tip of the catheter is positioned adjacent to targeted tissue, at which time energy is delivered to create tissue necrosis, rendering the ablated tissue incapable of conducting electrical signals. The dose of energy delivered is a critical factor in increasing the likelihood that the treated tissue is permanently incapable of electrical conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis, while avoiding excessive energy leading to collateral tissue injury.

Atrial fibrillation (AF), one of the most common arrhythmias, can be treated using balloon cryotherapy. In the earliest stages of the disease, paroxysmal AF, the treatment strategy involves isolating the pulmonary vein(s) from the left atrial chamber of the heart. Recently, the use of balloon cryotherapy procedures to treat AF has increased. In part, this stems from ease of use, shorter procedure times, and improved patient outcomes. Ablation of the muscle tissue, located in the atrial chamber of the heart, which is adjacent to the ostium (or opening) of the pulmonary vein can be accomplished using cryoballoon ablation therapy. When a cryoballoon is used during a pulmonary vein isolation (PVI) procedure, it is important that the cryoballoon completely occludes blood flow from the pulmonary vein to be isolated. If this is the case, then the application of cryo energy could reasonably result in electrically isolating the pulmonary vein.

Cryoballoon catheters (also sometimes referred to herein as “balloon catheters”) typically include an inner balloon and an outer balloon that encircles the inner balloon. The inner balloon and the outer balloon define an inter-balloon space between the inner balloon and the outer balloon. The inner balloon is selectively in fluid communication with a high pressure cryogenic fluid line (hereinafter sometimes referred to as a “high pressure fluid line”), in which a cryogenic fluid is injected into the inner balloon. The outer balloon, which surrounds the inner balloon, generally protects the patient by capturing and/or retaining the cryogenic fluid should the inner balloon rupture during a cryoablation procedure. Accordingly, pressure buildup within the inter-balloon space should be avoided to inhibit rupture of the outer balloon during the cryoablation procedure. If excessive pressure occurs in the inter-balloon space and rupturing of the outer balloon occurs, the health of the patient would be put at significant risk since the cryogenic fluid could expel into the patient's blood stream.

### SUMMARY

The present invention is directed toward a pressure inhibitor for an intravascular catheter system. The intravascular catheter system includes a handle assembly, an inner balloon, an outer balloon and a low pressure fluid line. The inner balloon and the outer balloon define an inter-balloon space therebetween. The low pressure fluid line extends between the handle assembly and the inter-balloon space. The low pressure fluid line is in fluid communication with the inter-balloon space. In certain embodiments, the pressure inhibitor includes a check valve that is positioned on the low pressure fluid line. The check valve inhibits flow of a fluid to the inter-balloon space.

In various embodiments, the check valve can be positioned within the handle assembly. Alternatively, the check valve can be positioned outside the handle assembly. In some such embodiments, the check valve can be positioned between the handle assembly and the inner-balloon space.

In certain embodiments, the pressure inhibitor can also include a pressure relief valve. In some such embodiments, the pressure relief valve is positioned on the low pressure fluid line, and can release pressure within the low pressure fluid line. In various embodiments, the pressure relief valve can be positioned within the handle assembly. Alternatively, the pressure relief valve can be positioned outside the handle assembly. In some such embodiments, the pressure relief valve can be positioned between the handle assembly and the inner-balloon space.

In another embodiment, the present invention is directed toward a pressure inhibitor for an intravascular catheter system. The intravascular catheter system includes a handle assembly, an inner balloon, an outer balloon and a low pressure fluid line. The inner balloon and the outer balloon define an inter-balloon space therebetween. The low pressure fluid line extends between the handle assembly and the inter-balloon space. The low pressure fluid line is in fluid communication with the inter-balloon space. In certain embodiments, the pressure inhibitor includes a pressure relief valve that is positioned on the low pressure fluid line. The pressure relief valve inhibits flow of a fluid to the inter-balloon space.

In certain embodiments, the pressure relief valve can be positioned within the handle assembly. Alternatively, the pressure relief valve can be positioned outside the handle assembly. In some such embodiments, the pressure relief valve can be positioned between the handle assembly and the inner-balloon space.

The present invention is also directed toward an intravascular catheter system. In certain embodiments, the intravascular catheter system includes a handle assembly, an inner balloon, an outer balloon that substantially encircles the inner balloon to define an inter-balloon space therebetween, a low pressure fluid line, and a pressure inhibitor. The low pressure fluid line extends between the handle assembly and the inter-balloon space. The low pressure fluid line is in fluid communication with the inter-balloon space. The pressure inhibitor is positioned on the low pressure fluid line. The pressure inhibitor inhibits flow of a fluid to the inter-balloon space.

In various embodiments, the pressure inhibitor includes a check valve. Additionally, or in the alternative, the pressure inhibitor can include a pressure relief valve. In some embodiments, the pressure inhibitor can be positioned within the handle assembly. Additionally, or in the alternative, the pressure inhibitor can be positioned outside the handle assembly. In some such embodiments, the pressure inhibitor can be positioned between the handle assembly and the inner-balloon space.

The present invention is also directed toward a method of inhibiting flow of a fluid to the inter-balloon space. The method includes positioning a pressure inhibitor on the low pressure fluid line, as described herein.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic view illustration of a patient and an embodiment of an intravascular catheter system having features of the present invention;

FIG. 2 is a simplified side view of a portion of an embodiment of the intravascular catheter system including one embodiment of a pressure inhibitor;

FIG. 3 is a simplified side view of a portion of an embodiment of the intravascular catheter system including another embodiment of the pressure inhibitor;

FIG. 4 is a simplified side view of a portion of an embodiment of the intravascular catheter system including yet another embodiment of the pressure inhibitor; and

FIG. 5 is a simplified side view of a portion of an embodiment of the intravascular catheter system including still another embodiment of the pressure inhibitor.

### DESCRIPTION

Embodiments of the present invention are described herein in the context of a pressure inhibitor for an intravascular catheter system. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy are used to ablate diseased heart tissue. Examples of these various forms of energy can include radio frequency (RF), ultrasound, pulsed DC electric fields and/or laser energy, to name a few. The present invention is intended to be effective with any or all of these forms of energy, or any other suitable form of energy.

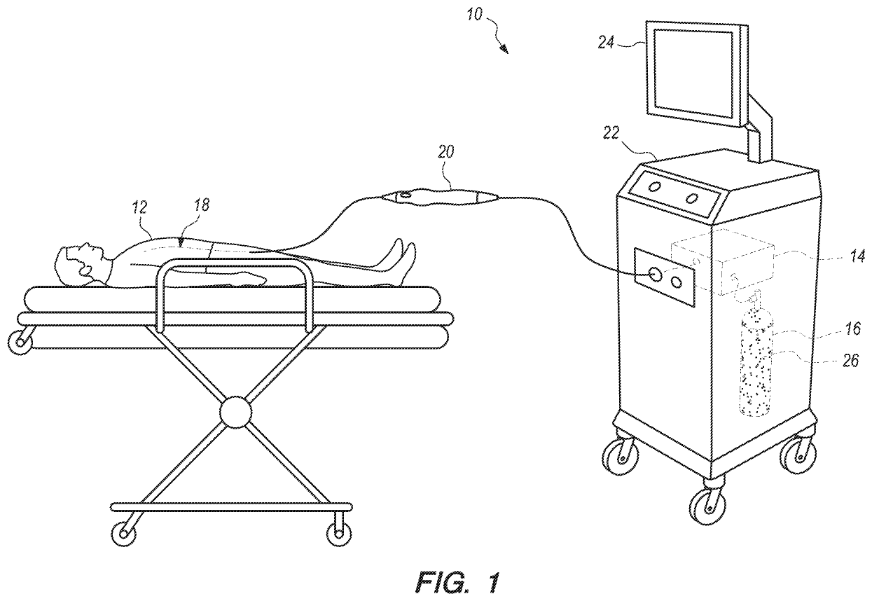

FIG. 1 is a simplified schematic side view illustration of an embodiment of an intravascular catheter system 10 for use with a patient 12, which can be a human being or an animal. The design of the intravascular catheter system 10 can be varied. In certain embodiments, such as the embodiment illustrated in FIG. 1, the intravascular catheter system 10 can include one or more of a controller 14 (illustrated in phantom), a fluid source 16 (illustrated in phantom), a balloon catheter 18, a handle assembly 20, a control console 22, and a graphical display 24.

It is understood that although FIG. 1 illustrates the structures of the intravascular catheter system 10 in a particular position, sequence and/or order, these structures can be located in any suitably different position, sequence and/or order than that illustrated in FIG. 1. It is also understood that the intravascular catheter system 10 can include fewer or additional components than those specifically illustrated and described herein.

In various embodiments, the controller 14 is configured to monitor and control various processes of the ablation procedure. More specifically, the controller 14 can monitor and control release and/or retrieval of a cooling fluid 26 (e.g., a cryogenic fluid) to and/or from the balloon catheter 18. The controller 14 can also control various structures that are responsible for maintaining and/or adjusting a flow rate and/or pressure of the cryogenic fluid 26 that is released to the balloon catheter 18 during the cryoablation procedure. In such embodiments, the intravascular catheter system 10 delivers ablative energy in the form of cryogenic fluid 26 to cardiac tissue of the patient 12 to create tissue necrosis, rendering the ablated tissue incapable of conducting electrical signals. Additionally, in various embodiments, the controller 14 can control activation and/or deactivation of one or more other processes of the balloon catheter 18. Further, or in the alternative, the controller 14 can receive data and/or other information (hereinafter sometimes referred to as “sensor output”) from various structures within the intravascular catheter system 10. In some embodiments, the controller 14 can receive, monitor, assimilate and/or integrate the sensor output and/or any other data or information received from any structure within the intravascular catheter system 10 in order to control the operation of the balloon catheter 18. As provided herein, in various embodiments, the controller 14 can initiate and/or terminate the flow of cryogenic fluid 26 to the balloon catheter 18 based on the sensor output. Still further, or in the alternative, the controller 14 can control positioning of portions of the balloon catheter 18 within the body of the patient 12, and/or can control any other suitable functions of the balloon catheter 18.

The fluid source 16 contains the cryogenic fluid 26, which is delivered to the balloon catheter 18 with or without input from the controller 14 during a cryoablation procedure. Once the ablation procedure has initiated, the cryogenic fluid 26 can be delivered and the resulting gas, after a phase change, can be retrieved from the balloon catheter 18, and can either be vented or otherwise discarded as exhaust. Additionally, the type of cryogenic fluid 26 that is used during the cryoablation procedure can vary. In one non-exclusive embodiment, the cryogenic fluid 26 can include liquid nitrous oxide. However, any other suitable cryogenic fluid 26 can be used. For example, in one non-exclusive alternative embodiment, the cryogenic fluid 26 can include liquid nitrogen.

The design of the balloon catheter 18 can be varied to suit the specific design requirements of the intravascular catheter system 10. As shown, the balloon catheter 18 is configured to be inserted into the body of the patient 12 during the cryoablation procedure, i.e. during use of the intravascular catheter system 10. In one embodiment, the balloon catheter 18 can be positioned within the body of the patient 12 using the controller 14. Stated in another manner, the controller 14 can control positioning of the balloon catheter 18 within the body of the patient 12. Alternatively, the balloon catheter 18 can be manually positioned within the body of the patient 12 by a healthcare professional (also referred to herein as an “operator”). As used herein, a healthcare professional and/or an operator can include a physician, a physician's assistant, a nurse and/or any other suitable person and/or individual. In certain embodiments, the balloon catheter 18 is positioned within the body of the patient 12 utilizing at least a portion of the sensor output that is received by the controller 14. For example, in various embodiments, the sensor output is received by the controller 14, which can then provide the operator with information regarding the positioning of the balloon catheter 18. Based at least partially on the sensor output feedback received by the controller 14, the operator can adjust the positioning of the balloon catheter 18 within the body of the patient 12 to ensure that the balloon catheter 18 is properly positioned relative to targeted cardiac tissue (not shown).

The handle assembly 20 is handled and used by the operator to operate, position and control the balloon catheter 18. The design and specific features of the handle assembly 20 can vary to suit the design requirements of the intravascular catheter system 10. In the embodiment illustrated in FIG. 1, the handle assembly 20 is separate from, but in electrical and/or fluid communication with the controller 14, the fluid source 16, and the graphical display 24. In some embodiments, the handle assembly 20 can integrate and/or include at least a portion of the controller 14 within an interior of the handle assembly 20. It is understood that the handle assembly 20 can include fewer or additional components than those specifically illustrated and described herein.

In various embodiments, the handle assembly 20 can be used by the operator to initiate and/or terminate the cryoablation process, e.g., start the flow of the cryogenic fluid 26 to the balloon catheter 18 in order to ablate certain targeted heart tissue of the patient 12. In certain embodiments, the controller 14 can override use of the handle assembly 20 by the operator. Stated in another manner, in some embodiments, the controller 14 can terminate the cryoablation process without the operator using the handle assembly 20 to do so.

The control console 22 is coupled to the balloon catheter 18 and the handle assembly 20. Additionally, in the embodiment illustrated in FIG. 1, the control console 22 includes at least a portion of the controller 14, the fluid source 16, and the graphical display 24. However, in alternative embodiments, the control console 22 can contain additional structures not shown or described herein. Still alternatively, the control console 22 may not include various structures that are illustrated within the control console 22 in FIG. 1. For example, in certain nonexclusive alternative embodiments, the control console 22 does not include the graphical display 24.

In various embodiments, the graphical display 24 is electrically connected to the controller 14. Additionally, the graphical display 24 provides the operator of the intravascular catheter system 10 with information that can be used before, during and after the cryoablation procedure. For example, the graphical display 24 can provide the operator with information based on the sensor output and any other relevant information that can be used before, during and after the cryoablation procedure. The specifics of the graphical display 24 can vary depending upon the design requirements of the intravascular catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

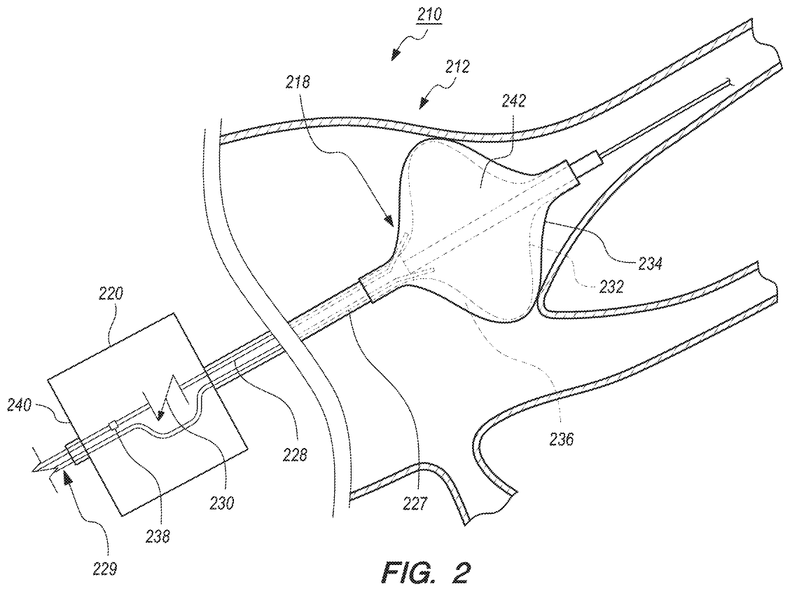

FIG. 2 is a simplified side view of a portion of an embodiment of the intravascular catheter system 210 including one embodiment of a pressure inhibitor 230. The controller 14 (illustrated in FIG. 1) and the cooling fluid source 16 (illustrated in FIG. 1) have been omitted from FIG. 2 for clarity. In the embodiment illustrated in FIG. 2, the intravascular catheter system 210 can include one or more of the balloon catheter 218, the handle assembly 220, a high pressure fluid line 227 (also referred to as a “fluid injection line”), a low pressure fluid line 228 (also referred to as a “fluid exhaust line”), an umbilical connector 229 and the pressure inhibitor 230.

The balloon catheter 218 is inserted into the body of the patient 212 during a cryoablation procedure. The design of the balloon catheter 218 can be varied to suit the design requirements of the intravascular catheter system 210. In this embodiment, the balloon catheter 218 includes an inner balloon 232 and an outer balloon 234. The outer balloon 234 substantially encircles the inner balloon 232. The outer balloon 234 can protect against cryogenic fluid 26 (illustrated in FIG. 1) from leaking out of the inner balloon 232 should the inner balloon 232 rupture or develop a leak during a cryoablation procedure. It is understood that the balloon catheter 218 can include other structures as well that are not shown and/or described relative to FIG. 2.

During use, the inner balloon 232 can be partially or fully inflated so that at least a portion of the inner balloon 232 expands toward and/or against a portion of the outer balloon 234 (although a space is shown between the inner balloon 232 and the outer balloon 234 in FIG. 2 for clarity). In this embodiment, the inner balloon 232 and the outer balloon 234 define an inter-balloon space 236 that is between the inner balloon 232 and the outer balloon 234.

The handle assembly 220 enables the operator or other user to operate, steer, position and control the balloon catheter 218. The design and specific features of the handle assembly 220 can vary. In this embodiment, the handle assembly 220 can include a pressure sensor 238 and an umbilical receptacle 240. It is understood that the handle assembly 220 can include fewer or additional components than those specifically illustrated and described herein.

In various embodiments, the pressure sensor 238 can measure and/or monitor the pressure within the low pressure fluid line 228, i.e., sense leaks and/or excessive pressure during cryoablation procedures. In other embodiments, the pressure sensor 238 can measure and/or monitor a balloon pressure in the inter-balloon space 236. As used in this embodiment, the “balloon pressure” means the pressure within the inter-balloon space 236 at or substantially contemporaneously with the time the pressure in the inter-balloon space 236 is measured. While in this embodiment the pressure sensor 238 is located on the low pressure fluid line 228 within the handle assembly 220, it is appreciated that the pressure sensor 238 can be located outside of the handle assembly 220, i.e., at any other suitable location within the intravascular catheter system 210.

In certain embodiments, the umbilical receptacle 240 provides connectivity between the handle assembly 220 and the umbilical connector 229. The design and specific features of the umbilical receptacle 240 can vary. As illustrated in FIG. 2, the umbilical receptacle 240 can contain a portion of the high pressure fluid line 227 and/or the low pressure fluid line 228. In this embodiment, the umbilical receptacle 240 can receive the umbilical connector 229 to provide electrical and/or fluid connectivity to the handle assembly 220. Alternatively, the umbilical connector 229 can be connected to the handle assembly 220 by any other method known to those skilled in the art.

The high pressure fluid line 227 is in fluid communication with an inner balloon interior 242 of the inner balloon 232. In certain embodiments, the high pressure fluid line 227 can include a relatively small diameter tube through which the cryogenic fluid 26, e.g., nitrous oxide, moves. In various embodiments, the high pressure fluid line 227 can allow the cryogenic fluid 26 to flow at any suitable pressure known to those skilled in the art sufficient to inject cryogenic fluid 26 into the inner balloon 232. In the embodiment illustrated in FIG. 2, a portion of the high pressure fluid line 227 is shown to extend from the umbilical connector 229 to the inner balloon interior 242.

In the embodiment illustrated in FIG. 2, the low pressure fluid line 228 is in fluid communication with the inter-balloon space 236. In various embodiments, the low pressure fluid line 228 can include a relatively small diameter tube that can provide the balloon pressure within the inter-balloon space 236 directly to the pressure sensor 238. Alternatively, the low pressure fluid line 228 can be connected to a vacuum (not shown). In such alternative embodiments, the low pressure fluid line 228 can function as a conduit through which fluid within the inter-balloon space 236 can be removed as exhaust from the balloon catheter 218. In the embodiment illustrated in FIG. 2, a portion of the low pressure fluid line 228 is shown to extend from the umbilical connector 229 to the inter-balloon space 236.

The umbilical connector 229 provides connectivity to the handle assembly 220. The design of the umbilical connector 229 can be varied to suit the design requirements of the intravascular catheter system 210. In various embodiments, the umbilical connector 229 can contain a portion of the high pressure fluid line 227 and the low pressure fluid line 228. In the embodiment illustrated in FIG. 2, the umbilical connector 229 can be connected to the umbilical receptacle 240 to provide connectivity of the high pressure fluid line 227 and the low pressure fluid line 228 to the handle assembly 220 within the intravascular catheter system 210. Alternatively, the umbilical connector 229 can be connected to the handle assembly 220 by any other method known to those skilled in the art. In the event that the umbilical connector 229 is not connected properly to the handle assembly 220, i.e., via the umbilical receptacle 240, the cryogenic fluid 26 contained within the high pressure fluid line 227 could potentially leak and/or flow into the low pressure fluid line 228.

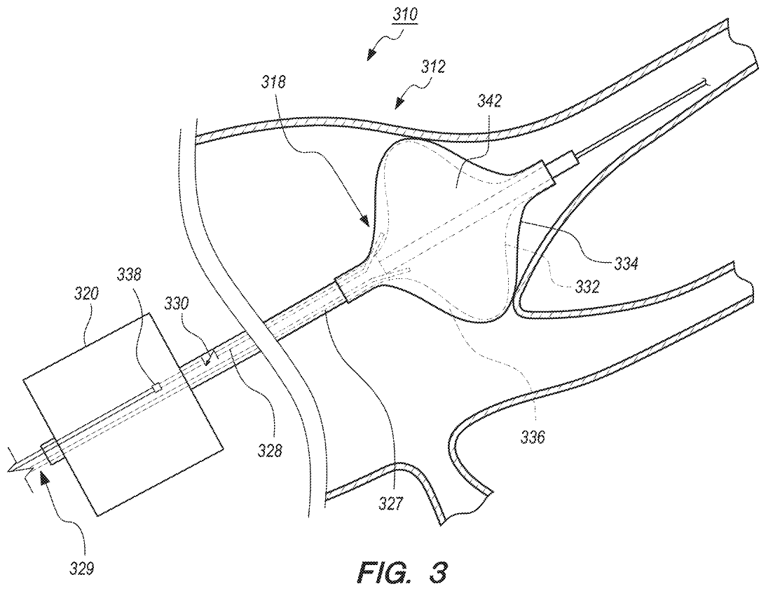

In various embodiments, the pressure inhibitor 230 can inhibit the flow of cryogenic fluid 26 (or any other fluid) toward the inter-balloon space 236 via the low pressure fluid line 228. The design and specific features of the pressure inhibitor 230 can vary. In the embodiment illustrated in FIG. 2, the pressure inhibitor 230 includes a check valve. The check valve only allows flow of fluid in a direction from the inter-balloon space 236 toward the handle assembly 220. In the event of a leak in the high pressure line 227 due to an improper connection or any problem that could cause the cryogenic fluid 26 to leak into the low pressure fluid line 228 during the cryoablation procedure, i.e., from the handle assembly 220, the umbilical connector 229 and/or umbilical receptacle 240, the pressure inhibitor 230 can inhibit the flow of fluid or excessive pressure from entering the inter-balloon space 236 via the low pressure fluid line 228. In the embodiment illustrated in FIG. 2, the pressure inhibitor 230 is positioned on the low pressure fluid line 228. In one nonexclusive embodiment, the pressure inhibitor 230 can be positioned within the handle assembly 220. In other non-exclusive embodiments, the pressure inhibitor 230 can be positioned outside the handle assembly 220 (illustrated in FIG. 3). In such embodiments, the pressure inhibitor 230 can be positioned between the handle assembly 220 and the outer balloon 234. Alternatively, the pressure inhibitor 230 can be positioned at any other suitable location on the low pressure fluid line 228. [0043] FIG. 3 is a simplified side view of a portion of an embodiment of the patient 312 and an intravascular catheter system 310 including another embodiment of a pressure inhibitor 330. The controller 14 (illustrated in FIG. 1) and the cooling fluid source 16 (illustrated in FIG. 1) have been omitted from FIG. 3 for clarity. In the embodiment illustrated in FIG. 3, the intravascular catheter system 310 includes the balloon catheter 318, the handle assembly 320, the high pressure fluid line 327 which extends into the inner balloon interior 342, the low pressure fluid line 328, the umbilical connector 329 and the pressure sensor 338, which are substantially the same structures and operate in substantially the same manner as those described with respect to FIG. 2.

FIG. 3 is a simplified side view of a portion of an embodiment of the patient 312 and an intravascular catheter system 310 including another embodiment of a pressure inhibitor 330. The controller 14 (illustrated in FIG. 1) and the cooling fluid source 16 (illustrated in FIG. 1) have been omitted from FIG. 3 for clarity. In the embodiment illustrated in FIG. 3, the intravascular catheter system 310 includes the balloon catheter 318, the handle assembly 320, the high pressure fluid line 327 which extends into the inner balloon interior 342, the low pressure fluid line 328, the umbilical connector 329 and the pressure sensor 338, which are substantially the same structures and operate in substantially the same manner as those described with respect to FIG. 2.

In the embodiment illustrated in FIG. 3, the pressure inhibitor 330 includes a check valve. In this embodiment, however, the pressure inhibitor 330 is positioned outside the handle assembly 320, along the low pressure fluid line 328 between the handle assembly 320 and the outer balloon 334. Alternatively, the pressure inhibitor 330 can be positioned in any other suitable location along the low pressure fluid line 328, such as between the handle assembly 320 and the control console 22 (illustrated in FIG. 1), as one non-exclusive example.

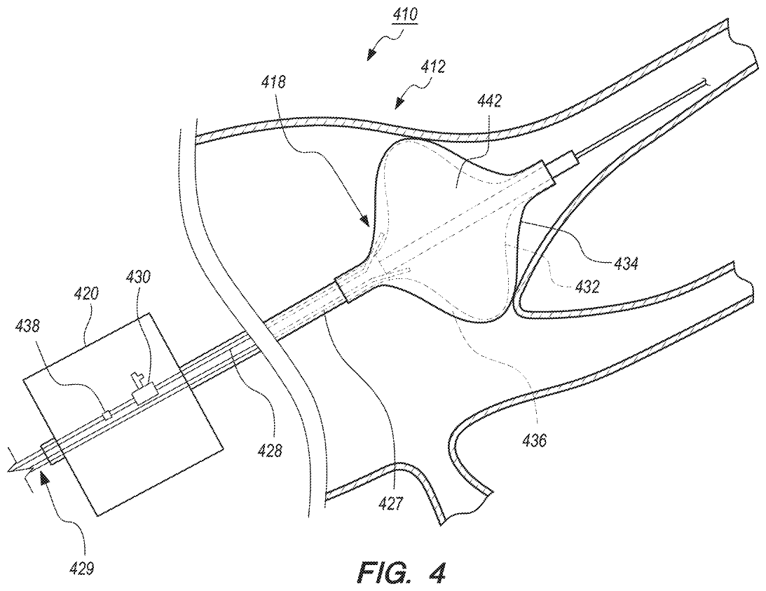

FIG. 4 is a simplified side view of a portion of an embodiment of the patient 412 and an intravascular catheter system 410 including yet another embodiment of a pressure inhibitor 430. The controller 14 (illustrated in FIG. 1) and the cooling fluid source 16 (illustrated in FIG. 1) have been omitted from FIG. 4 for clarity. In the embodiment illustrated in FIG. 4, the intravascular catheter system 410 includes the balloon catheter 418, the handle assembly 420, the high pressure fluid line 427 which extends into the inner balloon interior 442, the low pressure fluid line 428, the umbilical connector 429 and the pressure sensor 438, which are the substantially the same structures which operate in substantially the same manner as those described with respect to FIG. 2.

In various embodiments, the pressure inhibitor 430 can inhibit the flow of cryogenic fluid 26 (or any other fluid) toward the inter-balloon space 436 via the low pressure fluid line 428. The design and specific features of the pressure inhibitor 430 can vary. In the embodiment illustrated in FIG. 4, the pressure inhibitor 430 includes a pressure relief valve. The pressure relief valve can control pressure by allowing fluid in the inter-balloon space 436 and/or the low pressure fluid line 428 to be expelled as exhaust via at least a portion of the low pressure fluid line 428. In various embodiments, the pressure inhibitor 430 can be set to open at a predetermined threshold pressure. The predetermined threshold pressure can vary depending on the design parameters of the intravascular catheter system 410. When the predetermined threshold pressure is exceeded, i.e., the cryogenic fluid 26 (or any other fluid) from the high pressure fluid line 427 that has leaked or otherwise flowed into the low pressure fluid line 428 due to an improper connection or any other problem, the pressure inhibitor 430 can be forced open to allow the excess pressure to be diverted as exhaust prior to reaching the inter-balloon space 436.

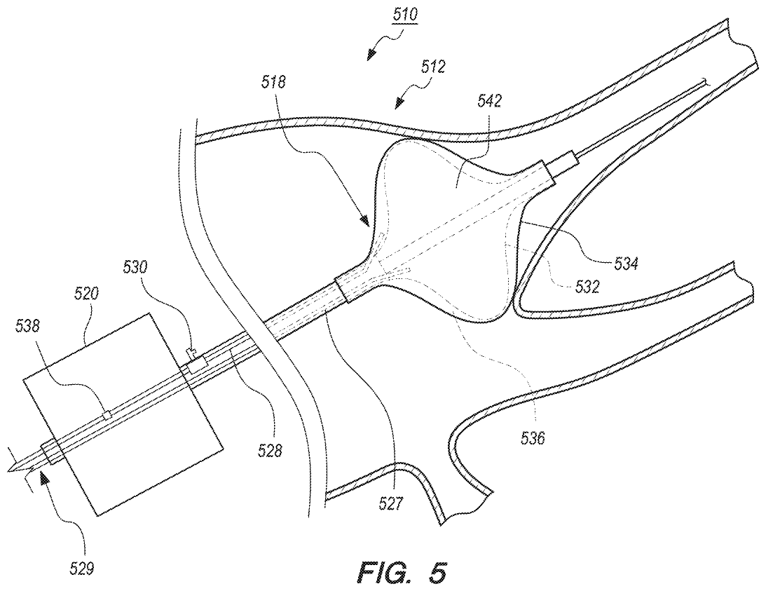

In this embodiment, the pressure inhibitor 430 is positioned on the low pressure fluid line 428. As one non-exclusive embodiment illustrated in FIG. 4, the pressure inhibitor 430 can be positioned within the handle assembly 420. In other non-exclusive embodiments, the pressure inhibitor 430 can be positioned outside the handle assembly 420 (illustrated in FIG. 5, as one non-exclusive example). In such embodiments, the pressure inhibitor 430 can be positioned between the handle assembly 420 and the outer balloon 434. Alternatively, the pressure inhibitor 430 can be positioned at any suitable location on the low pressure fluid line 428.

FIG. 5 is a simplified side view of a portion of an embodiment of the patient 512 and an intravascular catheter system 510 including still another embodiment of the pressure inhibitor 530. The controller 14 (illustrated in FIG. 1) and the cooling fluid source 16 (illustrated in FIG. 1) have been omitted from FIG. 5 for clarity. In the embodiment illustrated in FIG. 5, the intravascular catheter system 510 includes the balloon catheter 518, the handle assembly 520, the high pressure fluid line 527 which extends into the inner balloon interior 542, the low pressure fluid line 528, the umbilical connector 529 and the pressure sensor 538, which are substantially the same structures which operate in substantially the same manner as those described with respect to FIG. 4.

In the embodiment illustrated in FIG. 5, the pressure inhibitor 530 includes a pressure relief valve. In this embodiment, however, the pressure inhibitor 530 is positioned outside the handle assembly 520, along the low pressure fluid line 528 between the handle assembly 520 and the outer balloon 534.

It is appreciated that some or all of the embodiments of the pressure inhibitor 230, 330, 430, 530 described in detail herein can enable the realization of one or more certain advantages in the event of a leak of any cryogenic fluid 26 (or any other fluid) from the high pressure fluid line 227, 327, 427, 527 into the low pressure fluid line 228, 328, 428, 528 during a cryoablation procedure. With the various designs illustrated and described herein, the pressure inhibitor 230, 330, 430, 530 can help to protect from and/or reduce the likelihood of any cryogenic fluid 26 entering into the inter-balloon space 236, 436, via the low pressure fluid line 228, 328, 428, 528, which could cause the outer balloon 234, 334, 434, 534 to rupture.

It is understood that although a number of different embodiments of the pressure inhibitor have been illustrated and described herein, one or more features of any one embodiment can be combined with one or more features of one or more of the other embodiments, provided that such combination satisfies the intent of the present invention.

While a number of exemplary aspects and embodiments of the pressure inhibitor have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A pressure inhibitor for an intravascular catheter system, the intravascular catheter system including (i) a handle assembly, (ii) an inner balloon, (iii) an outer balloon and (iv) a low pressure fluid line, the inner balloon and the outer balloon defining an inter-balloon space therebetween, the low pressure fluid line extending between the handle assembly and the inter-balloon space, the low pressure fluid line being in fluid communication with the inter-balloon space, the pressure inhibitor comprising a check valve that is positioned on the low pressure fluid line, the check valve inhibiting flow of a fluid to the inter-balloon space.

2. The pressure inhibitor of claim 1 wherein the check valve is positioned within the handle assembly.

3. The pressure inhibitor of claim 1 wherein the check valve is positioned outside the handle assembly.

4. The pressure inhibitor of claim 3 wherein the check valve is positioned between the handle assembly and the inner-balloon space.

5. The pressure inhibitor of claim 1 further comprising a pressure relief valve, the pressure relief valve being positioned on the low pressure fluid line, the pressure relief valve releasing pressure within the low pressure fluid line.

6. The pressure inhibitor of claim 5 wherein the pressure relief valve is positioned within the handle assembly.

7. The pressure inhibitor of claim 5 wherein the pressure relief valve is positioned outside the handle assembly.

8. The pressure inhibitor of claim 7 wherein the pressure relief valve is positioned between the handle assembly and the inner-balloon space.

9. The pressure inhibitor of claim 1 further comprising a pressure relief valve, the pressure relief valve being positioned on the low pressure fluid line, the pressure relief valve releasing pressure within the inter-balloon space.

10. The pressure inhibitor of claim 9 wherein the pressure relief valve is positioned within the handle assembly.

11. The pressure inhibitor of claim 9 wherein the pressure relief valve is positioned outside the handle assembly.

12. The pressure inhibitor of claim 11 wherein the pressure relief valve is positioned between the handle assembly and the inner-balloon space.

13. A pressure inhibitor for an intravascular catheter system, the intravascular catheter system including (i) a handle assembly, (ii) an inner balloon, (iii) an outer balloon and (iv) a low pressure fluid line, the inner balloon and the outer balloon defining an inter-balloon space therebetween, the low pressure fluid line extending between the handle assembly and the inter-balloon space, the low pressure fluid line being in fluid communication with the inter-balloon space, the pressure inhibitor comprising a pressure relief valve that is positioned on the low pressure fluid line, the pressure relief valve releasing pressure within the inter-balloon space.

14. The pressure inhibitor of claim 13 further comprising a pressure relief valve, the pressure relief valve being positioned on the low pressure fluid line, the pressure relief valve releasing pressure within the low pressure fluid line.

15. The pressure inhibitor of claim 13 wherein the pressure relief valve is positioned within the handle assembly.

16. The pressure inhibitor of claim 13 wherein the pressure relief valve is positioned outside the handle assembly.

17. An intravascular catheter system, comprising:
a handle assembly;
an inner balloon;
an outer balloon that substantially encircles the inner balloon to define an inter-balloon space therebetween;
a low pressure fluid line that extends between the handle assembly and the inter-balloon space, the low pressure fluid line being in fluid communication with the inter-balloon space; and
a pressure inhibitor that is positioned on the low pressure fluid line, the pressure inhibitor inhibiting flow of a fluid to the inter-balloon space, wherein the pressure inhibitor includes a check valve and a pressure relief valve.

18. The intravascular catheter system of claim 17 wherein the pressure inhibitor is positioned within the handle assembly.

19. The intravascular catheter system of claim 17 wherein the pressure inhibitor is positioned outside the handle assembly.

20. The intravascular catheter system of claim 19 wherein the pressure inhibitor is positioned between the handle assembly and the inner-balloon space.
